(19) United States
(12) Patent Application Publication
(10) Pub. No.: US 2013/0138156 A1
(43) Pub. Date: May 30, 2013

(54) OSTEOSYNTHESIS DEVICE

(71) Applicant: Guillaume Derouet, La Turballe (FR)

(72) Inventor: Guillaume Derouet, La Turballe (FR)

(73) Assignee: NEOSTEO, Reze (FR)

(21) Appl. No.: 13/814,793

(22) PCT Filed: Sep. 2, 2010
(86) PCT No.: PCT/EP2010/062899

§ 371 Date: Feb. 7, 2013

(51) Int. Cl. A61B 17/80

(52) U.S. Cl. CPC A61B 17/8004
USPC 606/289

(57) ABSTRACT

The osteosynthesis device includes a plate for pressing against a bone and at least one threaded rod for holding the plate on the bone, the rod presenting a first thread for anchoring the rod in a bone and a second thread for co-operating with a tapped thread formed in the plate, the device being characterized in that the thread root of the second thread defines a body of cylindrical shape and in that the thread crest of the second thread defines a truncated cone.

The field of the invention is that of osteosynthesis techniques.

More precisely, the invention relates to an osteosynthesis device of the type comprising a plate for pressing against a bone and a threaded rod for holding bone fragments relative to the plate.

Osteosynthesis techniques are used to consolidate bone in certain fractures that require a device to be mounted on the bone fragments, the device being designed to stabilize and prevent excessive inter-fragment movement.

In a conventional technique, an osteosynthesis device comprises:
a support or plate presenting at least one hole with a tapped thread, the plate being designed to be pressed appropriately against bone fragments with the best possible shape match or “congruence”; and
at least one rod presenting at its tip a first thread for anchoring the rod in the bone, and presenting a second thread that it is to co-operate with a tapped thread in the plate so that once the rod is fully tightened, it clamps the plate against the bone (or the bone fragments).

The dimensions of the various elements making up the osteosynthesis device are relatively small in order to limit implantation trauma; in particular the diameter of the rod and the height of its threads are of small size.

Furthermore, while such devices are being put into place, it often happens that the rod and the tapped thread in the plate are found to have axes that are not identical, thereby running the risk of misaligning the threads and thus making it difficult or even impossible to tighten the rod properly in the plate.

In an attempt to remedy that drawback and/or to improve the congruence between the plate and the bone, proposals have been made to provide threaded rods that present a thread for co-operating with a tapped thread in the plate in such a manner that the thread root of the thread on the rod and the crest of said thread both present the shape of a frustoconical body.

Nevertheless, it is found in practice that such a rod gives rise to several drawbacks, and in particular:
in certain circumstances, tightening the rod on the plate gives rise to compression and/or deformation such that it becomes difficult or even impossible to remove the threaded rods after they have been tightened; and
tightening the threaded rod against the plate gives rise to a lag-screw effect that tends to pull the plate out towards the head of the rod, and consequently to space the plate too far from the bone.

A particular object of the invention is to mitigate those drawbacks of the prior art.

More precisely, an object of the invention is to propose an osteosynthesis device that makes it possible, after the threaded rods have been tightened, to remove the threaded rods that have been screwed to the plate, and to do so without making use of additional means.

Another object of the invention is to provide such an osteosynthesis device that ensures optimum congruence for the plate on the bone.

These objects and others that appear below, are achieved by the invention, which provides an osteosynthesis device comprising a plate for pressing against a bone and at least one threaded rod for holding the plate on the bone, said rod presenting a first thread for anchoring said rod in a bone and a second thread for co-operating with a tapped thread formed in said plate, the device being characterized in that the thread root of said second thread defines a body of cylindrical shape and in that the thread crest of said second thread defines a truncated cone.

With such an osteosynthesis device, the longitudinal tightening stresses of the rod on the plate act in such a manner that the plate is entrained progressively by the threaded rod penetrating into the bone until the plate is well pressed against the bone so as to obtain good congruence.

According to the invention, the second thread of the rod presents a nominal diameter that decreases regularly going from the screw head towards the tip of the rod, whereas the thread root diameter is constant, as in a standard screw.

As a result, while the rod is being tightened in the corresponding tapped thread in the plate, the radial stresses applied by the rod against the tapped thread in the plate are limited to a considerable extent. In particular, the risk that tightening the rod in the plate will lead to jamming is considerably reduced, compared with the assemblies made using prior art screws. The distance between the thread crest and the thread root in the second thread is greater than or equal to the distance presented by conventional screws, and it increases regularly up to a maximum that is reached at the end of the screw head, thereby limiting the stresses between the rod and the plate as tightening progresses.

Thus, the threaded rod of the invention is subjected to less deformation and avoids deforming the corresponding tapped thread in the plate. Such a threaded rod is therefore easier to remove after it has been tightened in the plate.

Advantageously, said second thread is obtained by removing the crest of a screw presenting a nominal diameter D and a pitch P, the cylindrical thread root of said second thread presenting a diameter d3 such that:

d3>D−1.2268×P.

In this way, the thread root diameter d3 of the second thread is greater than the thread root diameter D3 norm of a conventional thread, which in accordance with the ISO standard is calculated using the formula:

D3 norm=D−1.2268×P.

The strength of the threaded rod can thus be increased.

Furthermore, the socket that is provided in the head of the threaded rod in order to receive a tightening tool, e.g. a socket of the hexagonal type, and that is used when tightening the rod in the plate, can be made bigger. Thus, for a given size of threaded rod it is possible to use a tightening tool that is larger than in the prior art, thereby facilitating screwing and unscrewing operations in terms of torque.

Preferably, the thread root diameter d3 of said second thread is such that:

d3≦D−0.8872×P.

This characteristic gives rise to a thread root diameter for the second thread that remains limited, even if it is greater than the diameter D3 norm of a standard screw, thus obtaining tolerance to misalignment at the beginning of the screw engagement of the threaded rod in the plate, while avoiding interference between the thread root of the second thread and the crest of the tapped thread.

In an advantageous solution, the cone angle of the truncated cone as defined by the crest of the second thread lies in the range 1° to 60°, with the cone angle of said truncated cone preferably being 9°.

According to another advantageous characteristic, said tapped thread presents a crest defining a second truncated cone.

Preferably, the first thread of the threaded rod presents a first pitch, and the second thread presents a second pitch that is less than or equal to said first pitch.

Such a characteristic contributes to optimizing the pressing of the plate against the bone and to avoiding any lag-screw effect.

With such a threaded rod, the tightening of the rod in the plate takes place more progressively than does the anchoring of the rod in the bone.

In an advantageous embodiment, said rod presents a head surmounting said second thread, said head being suitable for being received completely in said plate and presenting a surface for coming into abutment against a surface of complementary shape formed in said plate.

Under such circumstances, said surface of said head and said surface of complementary shape formed in said plate presents a shape belonging to the following group:
a truncated sphere;
a truncated cone; and
annular and flat.

In an advantageous variant, said first thread is made up of two portions:
an end portion presenting a cylindrical thread; and
an intermediate portion extending between said end portion and said second thread, said intermediate portion presenting a conical thread.

This obtains optimized anchoring of the rod in the bone, because of the presence of the intermediate portion and of its conical thread. In addition, such a first portion (with the conical thread of the intermediate portion) optimizes avoidance of the above-mentioned lag-screw effect during tightening of the rod in the plate.

Other characteristics and advantages of the invention appear more clearly on reading the following description of a preferred embodiment of the invention, given by way of non-limiting illustration, and with reference to the accompanying drawings, in which:

FIG. 1 is a simplified diagrammatic view of a threaded rod of an osteosynthesis device of the invention;

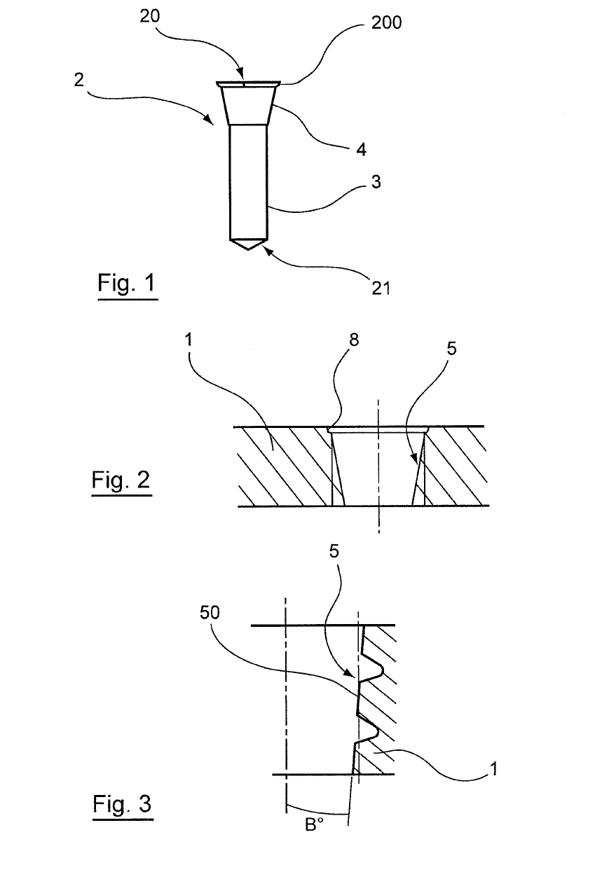

FIG. 2 is a simplified diagrammatic view of a plate of an osteosynthesis device of the invention;

FIG. 3 is a fragmentary section view of a tapped thread in a plate of an osteosynthesis device of the invention;

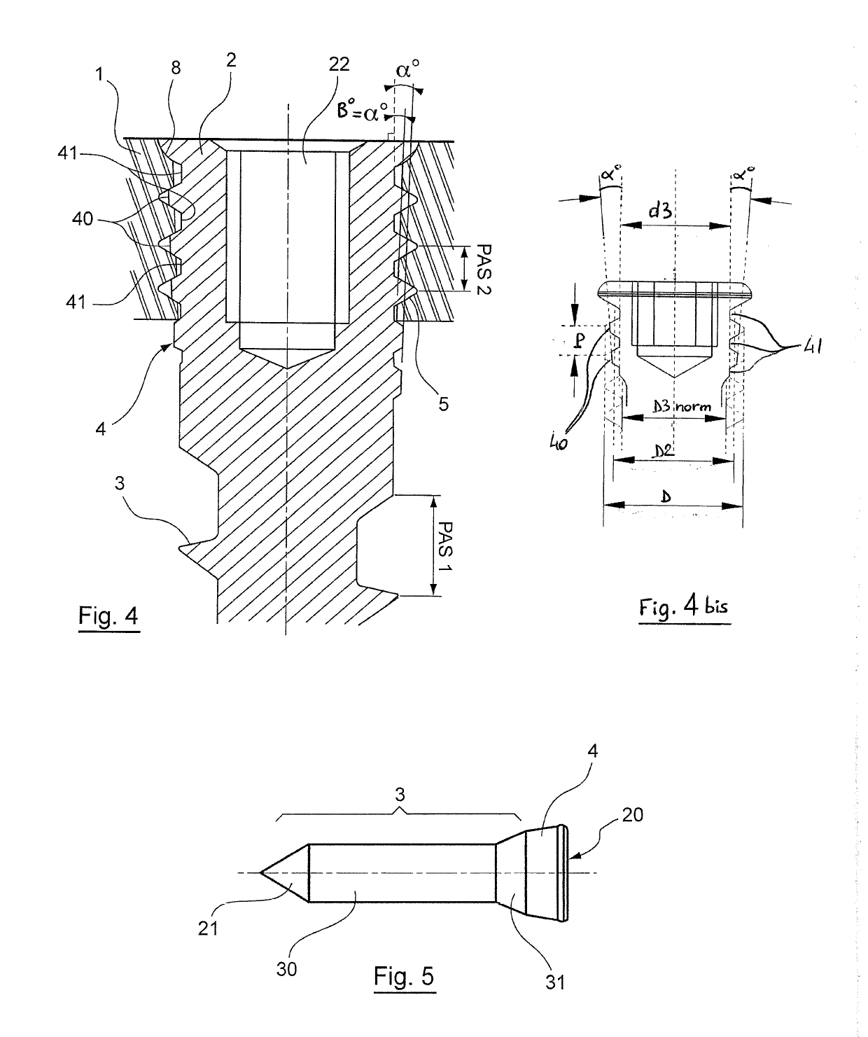

FIG. 4 is a view of a threaded rod screwed into a plate of an osteosynthesis device of the invention;

FIG. 4bis is a fragmentary view of a threaded rod in its portion including the second thread, in accordance with the invention; and

FIG. 5 is a view of a threaded rod of an osteosynthesis device in a variant embodiment of the invention.

As mentioned above, the principle of the invention consists in proposing an osteosynthesis device comprising a plate and at least one threaded rod, in which the threaded rod presents a thread for co-operating with the plate, the thread extending from a cylindrical body and the height of the thread decreasing going towards the base of the threaded rod.

With reference to FIGS. 1 and 2, a device of the invention comprises:
a plate 1, having at least one hole with a tapped thread 5; and
at least one threaded rod 2 presenting a screw head 20 and a tip 21 at its end opposite from the screw head, the threaded rod having two successive threads 3 and 4 between the tip 21 and the screw head 20.

In a portion extending from the tip 21 of the threaded rod, a first thread 3 is intended to anchor the rod in a bone (or in a bone fragment).

In a portion of the rod extending from the head 20, the threaded rod presents a second thread 4 for co-operating with a tapped thread 5 formed in the plate 1.

According to the principle of the invention and as shown in FIGS. 4 and 4bis, the second thread 4 extends radially from a thread root having a surface 41 defining a body of cylindrical shape, up to a thread crest 40 defining a truncated cone. In other words, the thread root of the second thread 4 defines a cylinder while the thread crest of the second thread 4 defines a frustoconical envelope.

By way of example, such a thread is made by machining a cylindrical body initially by making a conventional thread (of constant crest height). Thereafter, a second step is performed of machining the already-made thread so as to truncate it progressively in order to obtain a thread crest that defines a truncated cone with its larger diameter adjacent to the screw head.

This makes it possible to obtain a thread height that decreases regularly along the length of the second thread, going from the screw head towards the tip of the threaded rod.

It should be observed that machining the thread crest results in forming a flattened helical surface (instead of a projecting edge as is to be found with a conventional thread).

In FIG. 4bis, P designates the pitch and D the nominal diameter, prior to removal of the crest for the second thread 4 of the threaded rod.

In addition, D3 norm designates the diameter of the thread root of a screw having a nominal diameter D and a pitch P, in compliance with the ISO standard whereby:

D3 norm=D−1.2268×P.

Finally, D2 represents the diameter of the thread flanks and d3 represents the diameter of the thread root of the second thread 4.

In a preferred embodiment, the thread root diameter d3 of the second thread is such that:

d3>D3 norm; and

d3≦D−0.8872×P.

In a preferred embodiment, the cone angle α° of the truncated cone as defined by the crest of the second thread 4 is 9° (more generally, this cone angle α° potentially lying in the range 1° to 60°).

In parallel, with reference to FIG. 3, the tapped threads 5 in the plate 1 may present a crest defining a second truncated cone of cone angle B° that is generally less than 60°.

In a particular embodiment, as shown in FIG. 4, the cone angle B° of the truncated cone defined by the tapped thread 5 is equal to the cone angle α° of the truncated cone defined by the crest of the second thread 4. Nevertheless, it is possible to envisage that the cone angle B° of the truncated cone defined by the tapped thread 5 is equal to 0°.

Furthermore, and as shown in FIG. 4, the first thread 3 of the threaded rod presents a pitch written PAS 1 and the second thread 4 of the threaded rod presents a pitch written PAS 2, with:

PAS 1≧PAS 2.

In a preferred embodiment, the second thread is a two-start thread. Thus, the apparent pitch:

PAS 2=(PAS 1)/2.

More generally, the second thread is an n-start thread so that:

PAS 2=(PAS 1)/n.

The threaded rod 2 conventionally presents a socket 22 of section designed to receive a tightening tool of complementary shape.

According to another characteristic of the invention, the screw head 20 of the threaded rod presents a terminal element 200 that is to be received completely within the plate 1 (as shown in FIG. 4), this terminal element presenting a surface that is designed to come into abutment against a surface 8 of complementary shape formed in the plate.

This surface of the terminal element 200, and the surface of the complementary shape formed in the plate may present a shape selected in particular from the following shapes:
a truncated sphere;
a truncated cone; and
annular and flat.

With reference to FIG. 5, there follows a description of a threaded rod of an osteosynthesis device in a variant of the invention.

As can be seen in this figure, the threaded rod has a screw head 20, and at its end remote from the screw head, it has a tip 21, the threaded rod in this variant presenting three successive threads between the tip 21 and the screw head 20.

In a portion of the rod extending from the head 20, the threaded rod presents a so-called “second” thread 4 that is to co-operate with a tapped thread formed in the plate. On the general principle of the invention, this second thread 4 extends from a body of cylindrical shape and presents a thread having a crest that defines a truncated cone.

Over a portion extending from the tip 21 of the threaded rod, the first thread 3 for anchoring the rod in a bone is made up of two portions 30 and 31.

The first portion 30, referred to as the end portion, extends from the tip 21 and presents a cylindrical thread.

The second portion 31, referred to as an intermediate portion, extends between the first portion 30 and the second thread 4. This intermediate portion presents a conical thread having a cone angle that is preferably 32°, and more preferably that lies in the range 10° to 60°.

It should be observed that this intermediate portion is made so as to provide a connection between the end portion of the first thread and the second thread. In other words, the smallest thread diameter and the smallest thread root diameter in the intermediate portion correspond to the thread diameter and the thread root diameter of the thread in the end portion. In addition, the greatest thread diameter and the greatest thread root diameter in the intermediate portion correspond to the smallest thread diameter and to the smallest thread root diameter of the second thread 4.

1. An osteosynthesis device comprising a plate for pressing against a bone and at least one threaded rod for holding the plate on the bone, said rod presenting a first thread for anchoring said rod in a bone and a second thread for co-operating with a tapped thread formed in said plate, the device being characterized in that the thread root of said second thread defines a body of cylindrical shape and in that the thread crest of said second thread defines a truncated cone.

2. An osteosynthesis device according to claim 1, characterized in that said second thread is obtained by removing the crest of a screw presenting a nominal diameter D and a pitch P, the cylindrical thread root of said second thread presenting a diameter d3 such that:
d3>D−1.2268×P.

3. An osteosynthesis device according to claim 2, characterized in that the thread root diameter d3 of said second thread is such that:
d3≦D−0.8872×P.

4. An osteosynthesis device according to claim 1, characterized in that the cone angle of said truncated cone lies in the range 1° to 60°.

5. An osteosynthesis device according to claim 4, characterized in that the cone angle of said truncated cone is 9°.

6. An osteosynthesis device according to claim 1, characterized in that said tapped thread presents a crest defining a second truncated cone.

7. An osteosynthesis device according to claim 1, characterized in that the first thread presents a first pitch, and the second thread presents a second pitch that is less than or equal to said first pitch.

8. An osteosynthesis device according to claim 1, characterized in that said rod presents a head surmounting said second thread, said head being suitable for being received completely in said plate and presenting a surface for coming into abutment against a surface of complementary shape formed in said plate.

9. An osteosynthesis device according to claim 8, characterized in that said surface of said head and said surface of complementary shape formed in said plate presents a shape belonging to the following group:
a truncated sphere;
a truncated cone; and
annular and flat.

10. An osteosynthesis device according to claim 1, characterized in that said first thread is made up of two portions:
an end portion presenting a cylindrical thread; and
an intermediate portion extending between said end portion and said second thread, said intermediate portion presenting a conical thread.

11. An osteosynthesis device according to claim 2, characterized in that the cone angle of said truncated cone lies in the range 1° to 60°.

12. An osteosynthesis device according to claim 3, characterized in that the cone angle of said truncated cone lies in the range 1° to 60°.

13. An osteosynthesis device according to claim 11, characterized in that the cone angle of said truncated cone is 9°.

14. An osteosynthesis device according to claim 12, characterized in that the cone angle of said truncated cone is 9°.

15. An osteosynthesis device according to claim 2, characterized in that said tapped thread presents a crest defining a second truncated cone.

16. An osteosynthesis device according to claim 2, characterized in that the first thread presents a first pitch, and the second thread presents a second pitch that is less than or equal to said first pitch.

17. An osteosynthesis device according to claim 2, characterized in that said rod presents a head surmounting said second thread, said head being suitable for being received completely in said plate and presenting a surface for coming into abutment against a surface of complementary shape formed in said plate.
